(19) United States
(12) Patent Application Publication
(10) Pub. No.: US 2025/0125006 A1
(43) Pub. Date: Apr. 17, 2025

(54) DETERMINING PROTEIN-TO-PROTEIN INTERACTIONS

(71) Applicant: George Mason University, Fairfax, VA (US)

(72) Inventors: Mohsin Saleet Jafri, Potomac, MD (US); Leena Nezamuldeen, Jeddah (SA)

(21) Appl. No.: 18/918,695

(22) Filed: Oct. 17, 2024

Related U.S. Application Data
(60) Provisional application No. 63/590,997, filed on Oct. 17, 2023.

(51) Int. Cl. G16B 15/30; G16H 20/10

(52) U.S. Cl. CPC G16B 15/30; G16H 20/10

(57) ABSTRACT

A method includes: receiving information containing a name of a target protein associated with a disease, a name of a variant of the target protein, and a type of mutation associated with a disease; deriving, based on the information, a plurality of lists comprising: a first list containing protein names, a second list containing names of the genetic variant and protein post-translational modification type, a third list containing domain name, and a fourth list containing region name; generating search queries based on combinations of contents of the lists; gathering a plurality of text descriptions which satisfy the search queries, where the text descriptions include descriptions of relations of the target protein with other proteins; and identifying, based on processing the text descriptions, a suggested drug for treating the disease, where the suggested drug is associated with at least one of the other proteins.

This application claims the benefit of and priority to U.S. Provisional Application No. 63/590,997, filed on Oct. 17, 2023, the entire contents of which are incorporated by reference herein.

### TECHNICAL FIELD

The present disclosure relates generally to protein-protein interactions, and more specifically, to determining biological pathways which are involved in protein-protein interactions and which FDA-approved drug may target the pathway.

### BACKGROUND

Understanding the impact of genetic variants on protein structure and function is crucial for elucidating disease mechanisms and developing targeted therapies. Genetic variants can affect protein structure and function through several mechanisms such as altering the protein folding, disrupting protein-protein interactions, and modulating proteins levels of expression and regulation. The relationship between genetic variants and disease is intricate, involving direct impacts on protein structure and function as well as interactions with environmental factors. Understanding these dynamics is helpful for advancing personalized medicine approaches that target specific genetic profiles for prevention and treatment strategies.

### SUMMARY

In accordance with aspects of the present disclosure, a processor-implemented method includes: receiving a name of a target protein associated with a disease, a name of a variant of the target protein, and a type of mutation associated with a disease; deriving, based on the name of the target protein, the name of the variant, and the type of mutation, a plurality of lists including: a first list including different writing styles of protein names, a second list including different writing styles of the genetic variant and protein post-translational modification (PTM) type, a third list including domain name if the genetic variant is positioned in a domain, and a fourth list including region name if the genetic variant is located in a region; generating a plurality of search queries based on combinations of contents of the plurality of lists; gathering a plurality of text descriptions which satisfy the plurality of search queries, where the plurality of text descriptions includes descriptions of the relation of the target protein with a plurality of other proteins; and identifying, based on processing the plurality of text descriptions, at least one suggested drug for treating the disease, where the at least one suggested drug is associated with at least one of the plurality of other proteins.

In embodiments of the processor-implemented method, the processor-implemented method further includes processing the plurality of text descriptions by applying a neural network to extract sentences containing protein-protein interactions (PPI).

In embodiments of the processor-implemented method, the neural network includes three layers of Bidirectional Long Short-Term Memory (BiLSTM) recurrent neural network (RNN) cells, and a BioWordVec pretrained word embedding layer to extract positive sentences that includes protein-protein interaction.

In embodiments of the processor-implemented method, the processor-implemented method further includes, in the extracted positive sentences, labeling the name of the target protein name with a first indicator and labeling the name of the other proteins that interact with the target protein with a second indicator.

In embodiments of the processor-implemented method, the labeling is performed by applying a named entity recognition (NER) model and using a conditional random fields (CRF) algorithm.

In embodiments of the processor-implemented method, the first indicator is a letter “P” and the second indicator is a letter “O”.

In embodiments of the processor-implemented method, the processor-implemented method further includes identifying, based on the first indicators and the second indicators in the labeled sentences, shortest paths between separate proteins described in the extracted sentences.

In embodiments of the processor-implemented method, the processor-implemented method further includes extracting relationship words in the extracted sentences relating to relationships of the separate proteins, where the extracting uses predetermined patterns.

In embodiments of the processor-implemented method, the processor-implemented method further includes creating a PPI network based on the separate proteins described in the extracted sentences and based on the relationship words in the extracted sentences relating to relationships of the separate proteins.

In embodiments of the processor-implemented method, the identifying the at least one suggested drug for treating the disease includes: analyzing expression levels of the plurality of other proteins; identifying at least one other protein of the plurality of other proteins having altered expression levels; and identifying the at least one suggested drug for treating the disease based on the at least one other protein having altered expression levels.

In embodiments of the processor-implemented method, the at least one suggested drug is not associated with the protein associated with the disease.

In embodiments of the processor-implemented method, wherein the variant of the protein includes an amino acid substitution.

In embodiments of the processor-implemented method, the amino acid substitution is located at a phosphorylation, acetylation, methylation, sumoylation, or ubiquitination site.

In embodiments of the processor-implemented method, the variant of the protein includes a truncated protein.

In embodiments of the processor-implemented method, the protein-protein interaction (PPI) network identifies an abnormal protein-protein interaction.

In accordance with aspects of the present disclosure, a system includes: one or more processors, and one or more processor-readable medium having stored thereon instructions. The instructions, when executed by the one or more processors, cause the system at least to perform any one of the processor-implemented methods described above or shown in the claims section, which shall be incorporated by reference herein into this section.

In accordance with aspects of the present disclosure, a processor-readable medium has stored thereon instructions which, when executed by one or more processors of a system, cause the system at least to perform any one of the processor-implemented methods described above or shown in the claims section, which shall be incorporated by reference herein into this section.

The details of one or more embodiments of the disclosure are set forth in the accompanying drawings and the description below. Other features, objects, and advantages of the techniques described in this disclosure will be apparent from the description and drawings, and from the claims.

### BRIEF DESCRIPTION OF FIGURES

In order to better understand the subject matter that is disclosed herein and to exemplify how it may be carried out in practice, embodiments will now be described, by way of non-limiting example, with reference to the accompanying drawings. With specific reference to the drawings, it is stressed that the particulars shown are by way of example and for purposes of illustrative discussion of embodiments of the disclosure.

FIG. 1A and FIG. 1B is a diagram of an example of an operation for determining protein-protein interactions (PPI) and suggested drugs based on PPI, in accordance with aspects of the present disclosure;

FIG. 2 is a diagram of an example of an operation for text mining to determine protein-protein interactions, in accordance with aspects of the present disclosure;

FIG. 3 is a diagram of an example of a Long Short-Term Memory (LSTM) cell, in accordance with aspects of the present disclosure;

FIG. 4 is a diagram of an example of LSTM cells in forward and backward layers of a recurrent neural network forming a BiLSTM recurrent neural network, in accordance with aspects of the present disclosure;

FIG. 5 is a diagram of an example of applying a shortest dependency path model to a sentence, in accordance with aspects of the present disclosure;

FIG. 6A and FIG. 6B is a diagram of an example of a protein-protein interaction (PPI) diagram, in accordance with aspects of the present disclosure;

FIG. 7 is a diagram of an example of an operation for text mining to determine a suggested drug, in accordance with aspects of the present disclosure; and

FIG. 8 is a block diagram of an example of components of a system, in accordance with aspects of the present disclosure.

### DETAILED DESCRIPTION

The present disclosure provides an innovative processor-implemented method to determine variant protein-protein interactions, which biological pathways are involved in said protein-protein interactions, and which FDA-approved drug targets the pathway. In an embodiment, the discovery of such protein-protein interactions provides treatment decisions and personalized medicine for subjects suffering from a specific disorder resulting from expression of a variant protein.

As described in detail below, the developed method is divided into four sections. The first component is data curation and mining on protein databases, which gives essential information about protein structure and function as well as determining the genetic variations in the proteins. The second step is to build a protein-protein interaction network by text-mining medical abstracts with natural language processing and artificial intelligence methods to find other proteins that interact with the defective protein. Third, the PPI is converted to a computational model such as a Boolean Model to determine the functional implication of the defective protein. The fourth step is to determine which medicinal products have been approved by the FDA as a treatment for the functional defect caused by proteins or other proteins affected by the defective protein. Finally, a report is created that includes the precise impact of the variant mutation on protein functions.

As used herein, variant proteins include, for example, amino acid substitutions leading to missense mutations (change in one amino acid), nonsense mutations (premature stop codon), frameshift mutations (addition or deletion of nucleotides not divisible by three, altering the reading frame), and insertion/deletion mutations (adding or removing a section of DNA), each potentially affecting protein structure and function. In one aspect, the amino acid substitution is located at a phosphorylation, acetylation, methylation, simulation, or ubiquitination site. In another aspect, the amino acid substitution may affect the corresponding wild type protein's normal protein-protein interactions within the cell. Such variant proteins are associated with a specific disease of interest.

The present disclose provides, through identification of protein-protein interaction networks, identification of cellular pathways within the cell in which the variant protein is associated. In another aspect the present disclosure provides a method for identification of alterations in the levels of protein expression within a cellular pathway which are associated with variant protein expression. Accordingly, the identification of PPI networks may be utilized to infer functional changes within a cellular pathway caused by expression of variant proteins within the pathway.

Such cellular pathways may serve as a model of complex molecular interactions among proteins within a cell that leads to a certain product or a change in the cell. Changes in the cellular pathway may lead to disease. In an embodiment the cellular pathway may be a signaling pathway. Such signaling pathways include but are not limited to the WNT, SHH, notch pathways and the MAPK, RAS, mTOR, JAK-STAT, and NF-κB signaling pathways. Abnormalities in said signaling pathways are known to be associated with specific diseases.

Diseases are often caused by variant proteins and FDA-approved drugs are developed and used to treat said diseases targeting the variant protein. In some instances, there are no FDA-approved drugs available that target the variant protein. Drugs may have limited effectiveness and/or toxic side effects because of a failure to selectively target the disease-causing variant protein. Accordingly, once protein protein-protein interactions have been identified within a cellular pathway, using the disclosed methods, FDA-approved drugs may be identified that target different proteins within the identified protein-protein interaction network, e.g., cellular pathway, and which may be used to treat the disease of interest.

In an embodiment, the diseases to be treated include any genetic disease in which a abnormal protein variant is known to be associated with said disease or disorder. Genetic diseases can be caused by a mutation in one gene, by mutations in multiple genes, or by a combination of gene mutations and environmental factors, all of which may lead to expression of a protein variant or abnormal changes in protein expression. Lists of genetic diseases to be treated include, but are not limited to, Down syndrome, Huntington's disease, Cystic fibrosis, Fragile X syndrome, Turner syndrome, Cancer, Diabetes, Duchenne muscular dystrophy, Haemophilia, Heart Disease, Familial hypercholesterolemia, Neurofibromatosis, Obesity, Sickle Cell Anemia, and Phenylketonuria (PKU) to name a few.

In the following description, certain specific details are set forth in order to provide a thorough understanding of disclosed aspects. However, one skilled in the relevant art will recognize that aspects may be practiced without one or more of these specific details or with other methods, components, materials, etc. In other instances, well-known structures associated with transmitters, receivers, or transceivers have not been shown or described in detail to avoid unnecessarily obscuring descriptions of the aspects.

Reference throughout this specification to “one aspect” or “an aspect” means that a particular feature, structure, or characteristic described in connection with the aspect is included in at least one aspect. Thus, the appearances of the phrases “in one aspect” or “in an aspect” in various places throughout this specification are not necessarily all referring to the same aspect. Furthermore, the particular features, structures, or characteristics may be combined in any suitable manner in one or more aspects.

Intracellular and extracellular proteins are the core building blocks of many intracellular signaling pathways. Protein-protein interactions (PPIs) describe the primary pathways for cell function and have been associated with disease development. Discovering the most recent form of interactions between proteins and other elements remains difficult.

With the emergence of Natural Language Processing (NLP) methods, PPI extraction from biomedical abstracts or other texts becomes feasible. The most challenging aspect of the job involves interpreting biological and biomedical language in order to obtain a meaningful explanation of all living things' complicated nature. Another challenge lies in the need to search through a large number of articles or texts to find an explanation for the cause of the disease, particularly in the case of complex diseases.

The PPI extraction can be interpreted using several different techniques, and many machine learning and deep learning techniques may be implemented. Kernel-based machine learning methods may attain good performance, but they require extensive feature engineering, including lexical and syntactic features. In contrast, the application of Neural Networks (NN) to learn the semantic features and structure of sentences in order to classify them may be an effective technique for PPI extractions because they do not require an extensive amount of feature engineering like the kernel-based method does. Extracting sentences with relationships between proteins' names in text may use Machine Learning (ML) and Deep Learning (DL) schemes, where deep learning methods may be more accurate and achieve greater performance. However, when both convolution neural networks (CNNs) in conjugation with recurrent neural networks (RNNs) are used to develop a model, this creates a model with a complicated structure that takes inordinate time in the training process, because whereas CNN exhibits a hierarchical structure, RNN exhibits a sequential structure.

Regarding another technique, the objectives of PPI mining can be illustrated as a binary classification problem to distinguish positive sentences from negative ones. Positive sentences would contain the names of proteins in conjunction with relationship words, while negative sentences would represent the opposite. Another technique is the Named Entity Recognition (NER) method. This method relies on features engineering and training on datasets so to recognize proteins' names in sentences and the relationship words.

In accordance with aspects of the present disclosure, disclosed is a comprehensive method for generating graph figures of PPI networks from only biomedical literature by combining various approaches mentioned above. The disclosed method is comprised of various phases including the development of DL models and the application of patterns to extract information from text and transfer it to a knowledge graph.

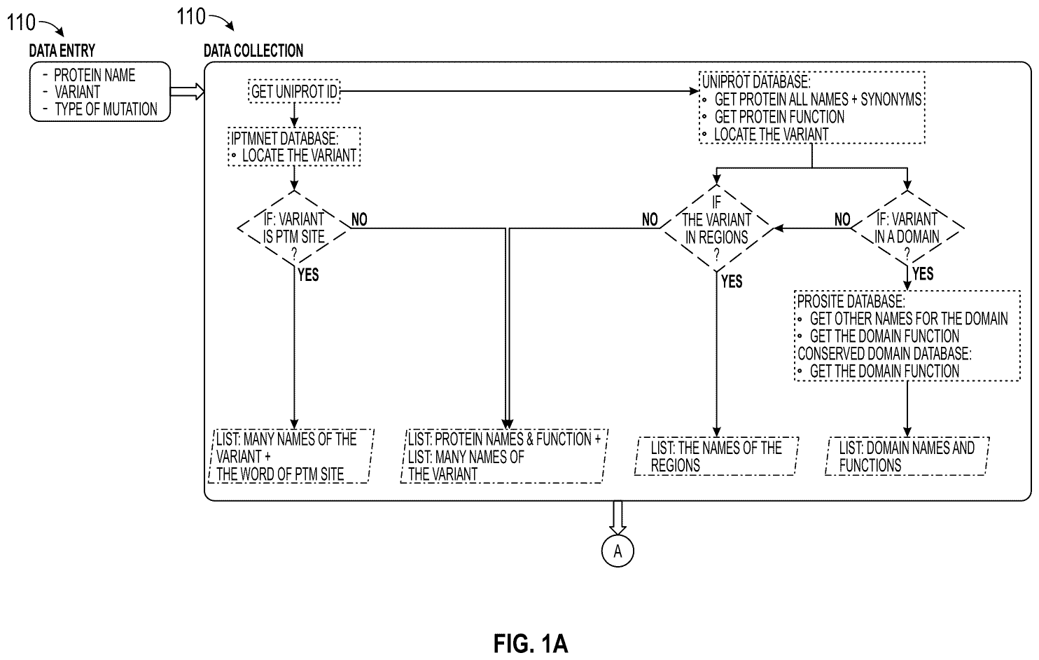

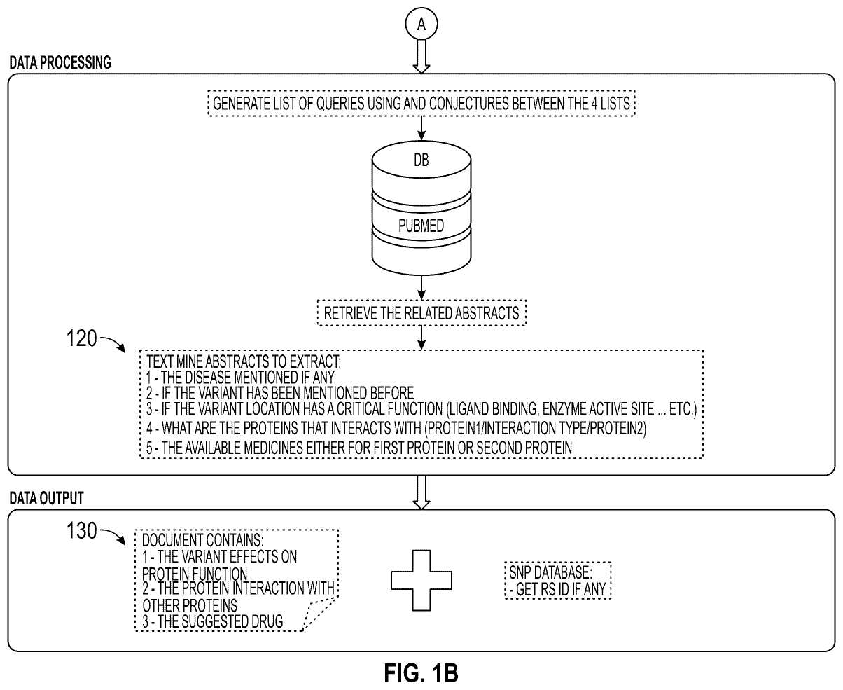

Referring to FIG. 1A and FIG. 1B, there is shown an example of the method, in accordance with aspects of the present disclosure.

A first phase 110 involves gathering information about the genetic variant location on the protein and the protein from public protein databases to understand which functional area of the protein is disrupted. Aspects of the first phase 110 involve accepting entry of three possible inputs: protein name, name of genetic variant (e.g., on the format of X0000X), and type of mutation. The first phase 110 may involve data extraction and collection. This phase can be implemented using Python or other programming languages.

More specifically, regarding the first phase 110, a database (e.g., UniProt database), can be accessed to determine other names of a protein, protein function, and variant location. If the variant is located in a domain, the process can extract information about the domain and its function from, e.g., PROSITE, and conserved domain databases. If the variant is located in a critical region, the process can extract the name of the region. Further, a database (e.g., iPTMnet database), can be accessed to extract whether the variant is a phosphorylation, acetylation, methylation, sumoylation, or ubiquitination site, and to extract whether the enzyme relates to the protein post-translational modification (PTM) type.

In aspects of the first phase 110, the data collection process includes the creation of four lists: a first list containing protein names and/or function and/or different writing styles of protein names (e.g., different ways that a protein can be named), a second list containing different writing styles for the genetic variant (e.g., names of genetic variants, or inclusion of the words “substitution OR mutation” to describe a variant) and/or PTM type if any, a third list containing domain name and function if the genetic variant is positioned in a domain, and a fourth list containing region name if the genetic variant is located in a region. The first phase involves generating search queries between the four lists using “AND” to use them as search terms to extract descriptions (e.g., abstracts of publications related to the protein or domain or region) from a data store (e.g., PubMed database).

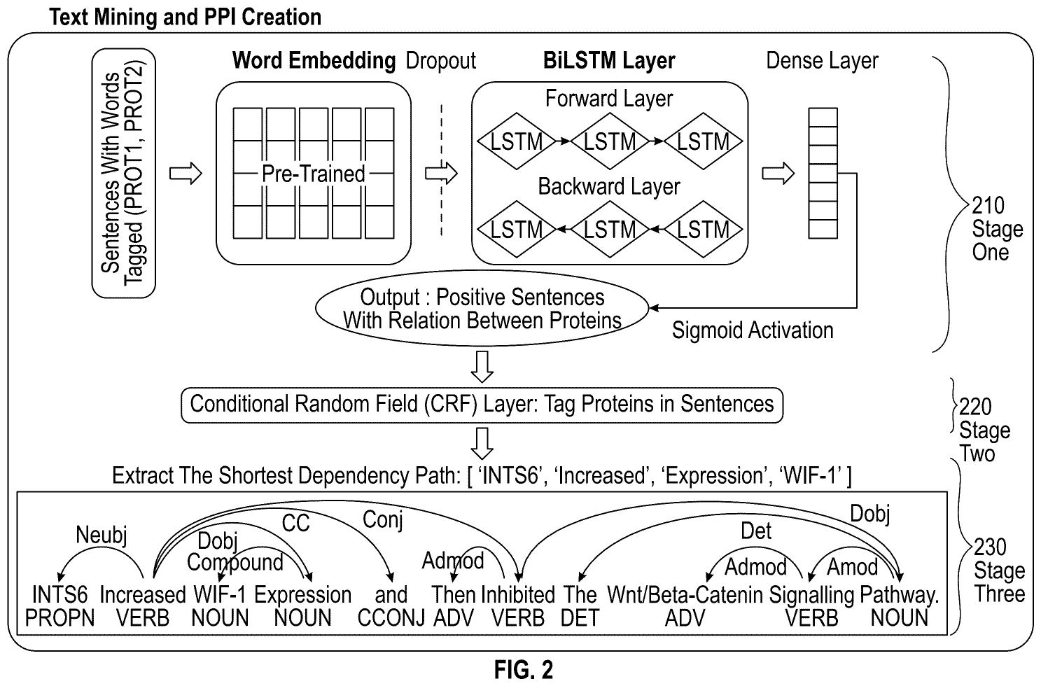

A second phase 120 involves, after gathering the descriptions (e.g., abstracts), a text mining method that creates a network that includes the mutated protein and the other proteins that interact with it, which will be referred to herein as a protein-protein interaction (PPI) network. This second phase 120 includes five stages, which will be described in more detail below in connection with FIGS. 2-6B.

A third phase 130 includes, after creating the PPI network, analysis using a Boolean Network Analysis (BNA) algorithm. Based on the expression levels of the proteins determined through this analysis, drug suggestions can be made. Proteins with altered expression levels can serve as input keywords in a drug database to identify available treatments. This process can be implemented using Python or other programming languages. Further aspects of the third phase will be described later herein. The output of the processing of FIG. 1A and FIG. 1B may be an output document as shown in FIG. 1A and FIG. 1B.

FIG. 1A and FIG. 1B is merely illustrative, and variations are contemplated to be within the scope of the present disclosure. For example, in various embodiments, other components or processes may be incorporated into the operation of FIG. 1A and FIG. 1B. In various embodiments, the operation of FIG. 1A and FIG. 1B may not include all of the components or processes shown in FIG. 1A and FIG. 1B. Such and other variations are contemplated to be within the scope of the present disclosure.

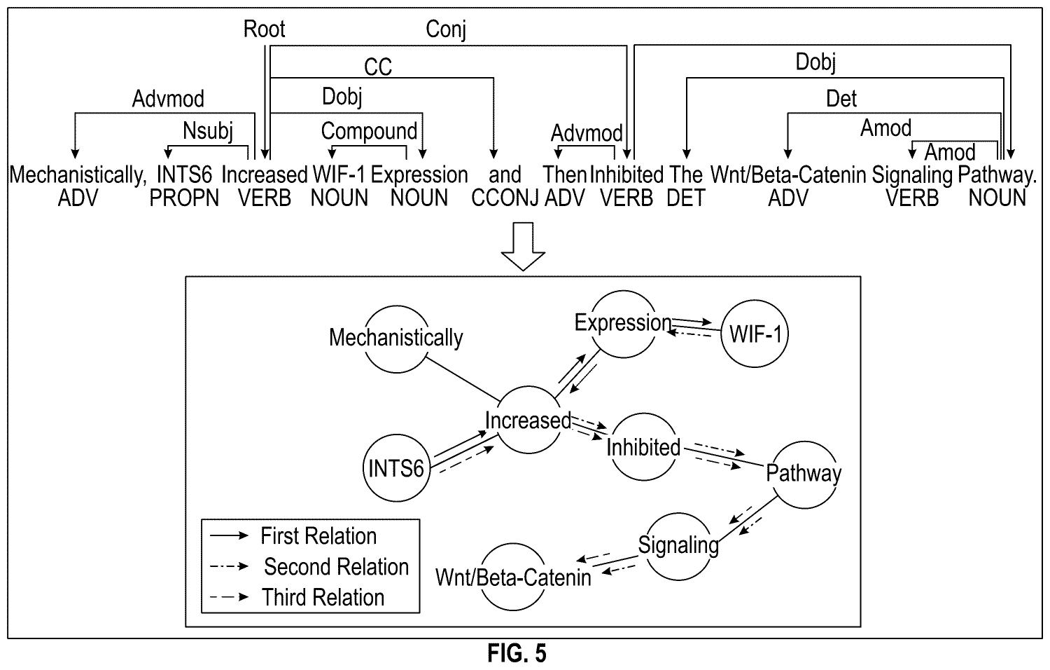

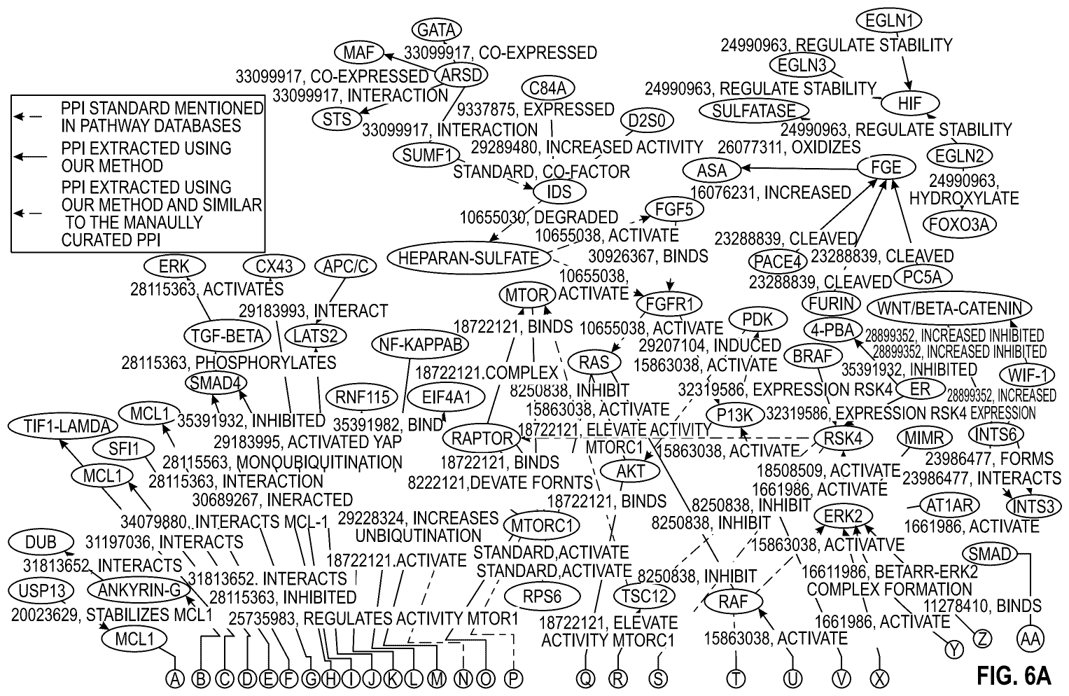

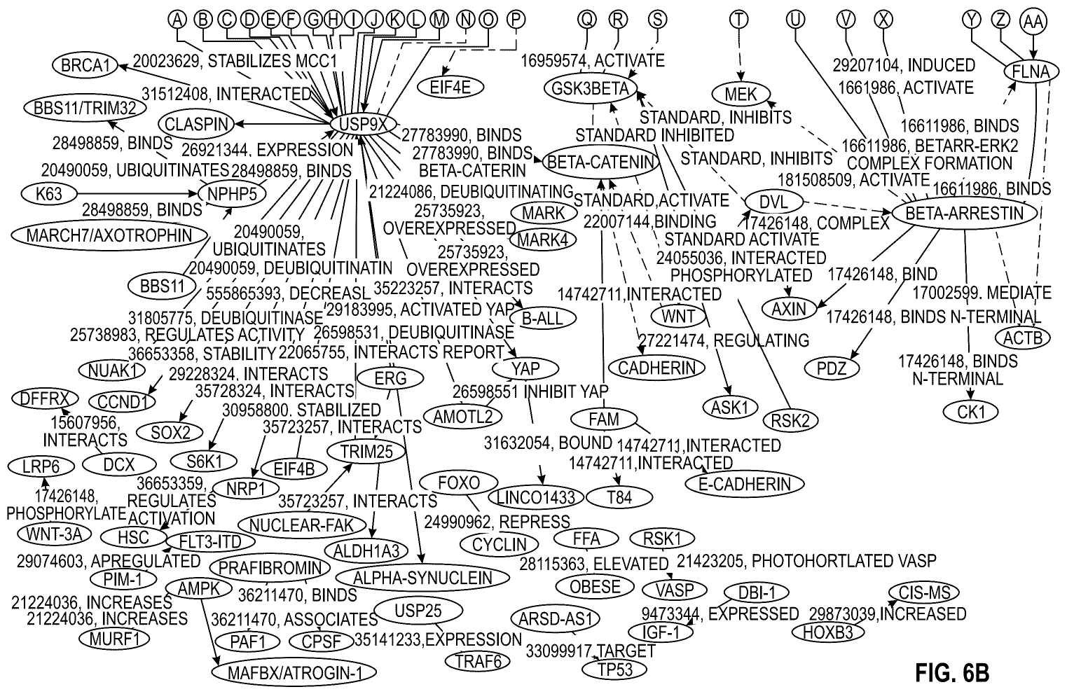

Referring to FIG. 2, there is shown further aspects of the second phase 120 of FIG. 1, including three of the five stages of second phase. The fourth stage will be described in connection with FIG. 5, and the fifth stage will be described in connection with FIG. 6A and FIG. 6B

In the first stage 210, a sentence classification model is created in order to distinguish between positive sentences containing protein relationships and negative sentences containing no protein links in biomedical abstracts. The output of the first stage 210 can be the positive sentences.

The first stage 210 involves a sentiment analysis method, which includes: creation of a neural network comprising three layers of BILSTM (Bidirectional Long Short-Term Memory) recurrent neural network (RNN) cells, along with a pretrained word embedding layer pretrained using, e.g., BioWordVec, which is a word embedding vector of 4 billion tokens. Such an architecture of three RNN layers combined with pretrained embeddings (e.g., BioWord Vec) has not been implemented before and is effective at extracting sentences that contain protein-protein interactions (PPI).

The method may take advantage of the AIMed and BioInfer corpora (both available at http://corpora.informatik.hu-berlin.de), which contain reference corpora for PPI extraction applications, to train the DL model. Furthermore, to expand the learning of semantic and syntactic features of the text, a pre-trained word embedding vector on more than 20 million biomedical documents from PubMed and more than four billion words of biomedical terms can be used for training the DL model. When a sentence contains the names of two proteins with a relationship between them, it is considered a positive sentence and is labeled 1; otherwise, the sentence is considered a negative sentence and is labeled 0.

In accordance with aspects of the present disclosure, the processing of the first stage 210 may involve text pre-processing. The model can be trained with Almed and BioInfer corpus data. The two datasets can be integrated and prepared for processing via a Python NLTK library. Combined, the two datasets have approximately 1060 abstracts and 3067 sentences. Two types of data processing can be performed; however multiword tokenization can be used to ensure that protein names are comprehended in their entirety. Multi-word tokenization, for instance, tokenizes Beta and catenin with a dash sign between them if the term beta-catenin is written in this way (Beta-catenin). The text pre-processing of data can include using two terms in place of actual protein names, e.g., “PROT1” and “PROT2.” These terms are meant to replace the first and second protein names in the sentences, respectively.

In aspects, the first stage 210 also involves word embedding. Word embedding is a representation learning technique comprising aligning words with similar meanings and convergingly representing them in a low-dimensional vector space. In the dataset, each word is represented as a vector of positive real values. Specifically, the publicly available pre-trained word embeddings BioWordVec and GloVe can be utilized in this model, with embedding representations of 4 billion tokens and 200-dimensional word embeddings, and 6 billion tokens and 200-dimensional word embeddings, respectively. When using Keras, these pre-trained word embedding models can be used to create a weight matrix for the embedding layer. Pre-trained word embedding, as opposed to one-hot encoding, which turns the words into binary vectors, reduces the distance between words with the same meaning and vectorizes them in real numbers. By minimizing the gap between the words, this strategy increases the coverage of words and makes it simpler to recognize the sentences containing information about the protein-protein interaction. On the other hand, one-hot encoding encodes two words with the same meaning in different real numbers. For example, the words (rise, increase) can be synonyms but have a different real number and not clustered together.

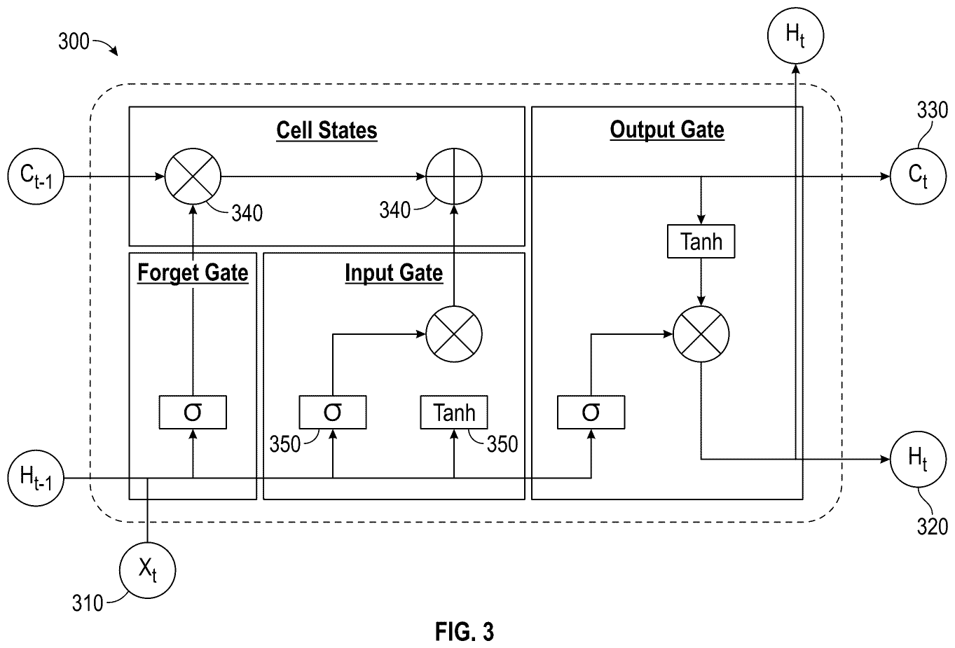

In aspects, the first stage 210 involves BILSTM Layers. Long Short-Term Memory (LSTM) artificial recurrent neural network (RNN) is useful in reducing the vanishing gradient mistakes and capturing the semantic information in long sentences because it is fast and efficient. Referring also to FIG. 3, each LSTM cell 300 has three gates: input gate, forget gate, and output gate. Each time iteration t, the cell 300 has layer input x 310 and layer output h 320. The C 330 is the input and the out of cell state. There are circles 340 containing arithmetic operations: multiplication and addition. The squares 350 are the gate activation function sigmoid, and tanh is the hyperbolic tangent function. The three gates with the cell states are controlling the learning route of the models.

During each time step, the quantity of information that travels through the neurons is controlled by the three gates. Forget gates are used to determine which of the previously hidden states should be reserved. Specifically, the forget gate enables the LSTM cell to be effective and scalable for a wide variety of sequential data feature learning. The input gate decides which of the currently hidden states should be retained. The cell state updates the cell states from the forget gate, output gate, and the input gate. The output gate decides the next hidden state.

Each LSTM cell's mathematical representation and the equations governing its three gates are as follows:

$${i}_{t =} σ ( {W}_{ix} {x}_{t} + {W}_{i h} {h}_{t - 1} + {b}_{i} ) \qquad ( 1 )$$

$${f}_{t =} σ ( {W}_{fx} {x}_{t} + {W}_{fh} {h}_{t - 1} + {b}_{f} ) \qquad ( 2 )$$

$${o}_{t} = σ ( {W}_{ox} + {W}_{o h} {h}_{t - 1} + {b}_{o} ) \qquad ( 3 )$$

$${c}_{t} = {f}_{t} * {c}_{t - 1} + {i}_{t} * tanh ( {W}_{cx} {x}_{t} + {W}_{c h} {h}_{t - 1} + {b}_{c} ) \qquad ( 4 )$$

$${h}_{t} = {o}_{t} * tanh ( {c}_{t} ) \qquad ( 5 )$$

where i is the input gate, f is the forget gate, σ is the output gate, c is the cell states, xₜ is the word embedding victors, hₜ is the hidden state, W is the weight matrices, b is the bias vectors, σ is the sigmoid function, tanh is the hyperbolic tangent function.

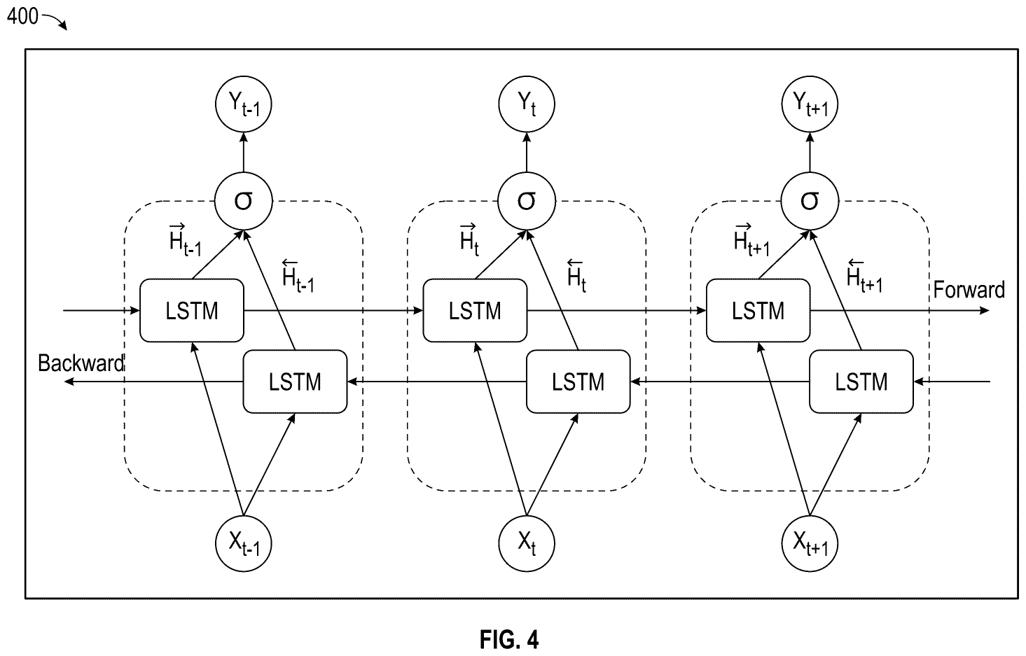

BiLSTM is well suited for use in sentiment analysis and text classification models. Referring also to FIG. 4, the LSTM cells in the forward and backward layers of the recurrent neural network form the structure of the BILSTM recurrent neural network 400. To obtain optimal performance, training on both the input sequence and its reverse duplicate is performed. The output of the word embedding layer is taken as input x. The BILSTM network 400 will train both the original sequence and its reversed counterpart. The sigmoid function will aggregate the result of both training directions and represent it as an output y.

As demonstrated in FIG. 4, xₜ is the word embedding victors. \vec{h} is the forward hidden layer, [symbol] is the backward hidden layer, and yₜ is the joining outputs from the forward and backward hidden layers. The output layer values processed as follows:

$${\vec{h}}_{t} = σ ( W {\to }_{xh} {x}_{t} + W {\to }_{hh} {\vec{h}}_{t - 1} {x}_{t} + b {\to }_{n} ) \qquad ( 6 )$$

$${\overleftarrow{h}}_{t} = σ ( W {←}_{xh} {x}_{t} + W {←}_{hh} {\overleftarrow{h}}_{t + 1} {x}_{t} + b {←}_{n} ) \qquad ( 7 )$$

$${y}_{t} = W {\to }_{hy} {\vec{h}}_{t} + W {←}_{hy} {\overleftarrow{h}}_{t} + {b}_{y} \qquad ( 8 )$$

where W is weights matrices, b is the bias term, σ is sigmoid function, and hₜ is the hidden state.

With continuing reference to FIG. 2, in the first stage 210, a dense layer is added at the end to ensure that all of the output neurons in the BILSTM neural network (e.g., FIG. 4, 400) were fully connected. Because of the usage of two classes for classification, positive and negative sentences, the output prediction of the model with a Softmax activation function is performed using the dense layer. This layer predicts a multinomial probability distribution. In this case, the prediction probability range is 0 to 1. A prediction of less than 0.5 is regarded as a negative prediction, whereas a prediction of equal to or greater than 0.5 is seen as a positive prediction but other threshold values may be used.

With continuing reference to FIG. 2, the second stage 220 involves a named entity recognition (NER) method. In Named Entity Recognition (NER) models, generative models such as Hidden Markov Models (HMM) have strict rules of learning that rely on the joint distribution of the data, which results in dependent features. Approaches such as pattern-based approach, one of the NLP methods, combines precision and complexity in its pattern design but this method requires that the extracted words or sentences must match the selected patterns. Although the pattern-based approach has limitations, the high precision of the outturns may make it useful in discovering the PPI relation words. The conditional Random Field (CRF) method is an effective approach for those types of tasks especially when learning from widely distributed data, such as the diversity in writing the contexts in biomedical literature. Discriminative models such as CRF rely on conditional functions with neighboring contextual consideration. This results in more efficient learning when learning from widely distributed data.

In aspects of the present disclosure, the second stage 220 involves developing a Named Entity Recognition (NER) model to label the protein names in sentences using the Conditional Random Field (CRF) method. This model output provides a tagging tool designed to find the protein names in the sentences.

The model is developed to tag the names of proteins after they have been extracted from positive sentences that describe relationships between proteins. The words in the sentences of each corpus are tokenized, position-tagged, and labeled. The P label is applied to the proteins mentioned in the text, whereas the O label is applied to everything else.

Text pre-processing for two datasets (Aimed/Bioinfer) can be performed and, using the letters (P) and (O) to label the protein names and other words, respectively, the datasets can be used to train the model (e.g., using sklearn-CRFSuite library in Python). Conditional Random Field (CRF) is a statistical probabilistic modelling used for structured prediction. Because there are only two labels (i.e., P and O), NER-CRF model may perform better than utilizing Neural Network (NN) models. The output of the trained model serves as a tagging tool to search and recognize the proteins names in sentences.

Following the selection of sentences containing relationships between proteins using the sentences classification model of the first stage, and the tagging of proteins names using the NER-CRF model of the second stage, the third stage 230 applies the shortest dependency path model to extract the shortest path between the names of the proteins in the selected positive sentences to extract relationships between the proteins. Interaction sentences in PPI are composed of nouns and verbs. The verbs are almost always the focal point of all sentences. Referring also to FIG. 5, dependency parsing illustrates the sentences as trees and recognizes and labels the center of the sentences as the ROOT of the tree, which is usually reflected in the verbs. The dependency labels for the remaining words are assigned by the shortest dependency path model based on the syntactic structure of the sentence.

With continuing reference to FIG. 2, the third stage 230 involves using the shortest dependency path algorithm to derive a pattern. This algorithm creates the shortest path between the two proteins related in the extracted sentences. The shortest dependency path algorithm may be implemented in Python or in other programming languages. By using the shortest dependency path model, the third stage 230 includes creating the patterns that will be used in the fourth stage to extract relationship words from PPI sentences. The third stage 230 assigns the dependency parsing labels for the words in the sentences and finds the shortest route between the names of the proteins in the sentences. The starting point of this route usually represents the relation words between the two proteins' names including sometimes other dependency labels. Patterns created to extract the relation words can be defined according to the dependency labels in the shortest path between the proteins' names in the sentences.

With reference to FIG. 5, the fourth stage involves using predetermined patterns with a matcher method to extract the relationship words in the sentences. For example, the sentence “Mechanistically INTS6 increased WIF-1 expression and then inhibited the Wnt/beta-catenin signaling pathway” has three tagged protein names: INTS6, WIFI, and Wnt/beta-catenin. There are a definite number of relations that can be extracted based on the number of protein names present, and in the example given in the preceding line, there are three relations that can be extracted, as shown in FIG. 5. Having collected the phrases with the shortest dependency paths, the dependency labels can be examined and investigated in order to identify a pattern that could be used to extract the accurate relationship. The first protein name dependency label is usually subj, the second protein name dependency label is usually obj. Discovering the range of dependency labels in which the relation words can be placed is part of the task. Usually, the ROOT dependency label (the verb in the middle of the sentence) defines the relation word but in some shortest dependency path sentences, the ROOT dependency label in conjugation with amod or dep dependency label is found to explain the relation words even more clearly. Other dependency labels were discovered to describe the relationships in the sentences, and these are taken into consideration as well. Thus, one or more patterns can be determined in this manner through analysis, and can be predetermined before applying the fourth stage.

The word with ROOT dependency label may locate and define the relationship between the two protein names in the sentences. The dependency label range of the first protein may be (‘nsubj’,‘amod’, ‘compound’), and the dependency label range of the second protein may be (‘dobj’,‘pobj’,‘npadvmod’,‘appos’). The predetermined patterns to locate and define the relationship words may be: (‘DEP’:‘amod’,‘OP’:“*”), (‘DEP’:‘conj’,‘OP’:“*”), (‘DEP’:‘ROOT’,‘OP’:“*”), and/or (‘DEP’:‘acomp’,‘OP’:“*”).

Usually, the ROOT dependency label (the verb in the middle of the sentence) is defining the relation word but in some shortest dependency path sentences, the ROOT dependency label in conjugation with amod or dep dependency label may explain the relation words even more clearly. Other dependency labels were discovered to describe the relationships in the sentences, and these were taken into consideration as well. The pattern was defined, and the relationship can be extracted using the shortest dependency path model and matcher library. The patterns may be:

```
pattern = [{‘DEP’:‘ROOT’,‘OP’:‘+’}],[{‘DEP’:‘acl’,‘OP’:‘{0}’}],
  [{‘DEP’:‘comp’,‘OP’:‘+’},{‘DEP’:‘acl’,‘OP’:‘!’}],
  [{‘DEP’:‘ROOT’,‘OP’:‘+’},{‘DEP’:‘npadvmod’,‘OP’:‘!’}],
  [{‘DEP’:‘ROOT’,‘OP’:‘+’},{‘DEP’:‘dep’,‘OP’:‘!’}],
  [{‘DEP’:‘ROOT’,‘OP’:‘+’},{‘DEP’:‘dobj’,‘OP’:‘!’}],
  [{‘DEP’:‘ROOT’,‘OP’:‘+’},{‘DEP’:‘amod’,‘OP’:‘!’}]].
```

A matcher library can be used to locate the relationship words from the sentences for which their dependency labels match the arrangement in the predetermined patterns.

Based on the predetermined patterns and the patterns created by the third stage, the fourth stage can match the predetermined patterns with the pattern created by the third stage to extract the relationship between the proteins in a sentence.

The fifth stage involves creating a PPI network incorporating the labeled proteins and their relationship terms, which can be implemented using Python or another programming language. An example of such a PPI network is shown in FIG. 6A and FIG. 6B. Any of the relationship words shown in FIG. 6A or FIG. 6B, or any other relationship words describing relationship of protein-protein interactions, are contemplated to be within the scope of the present disclosure.

FIGS. 2-6B and the description above are merely an example and variations are contemplated to be within the scope of the present disclosure. In various embodiments, the operation may include other blocks not shown or described in connection with FIGS. 2-6B. In various embodiments, the operation may not include every block shown or described in connection with FIGS. 2-6B. In various, the blocks may be performed in a different order than as shown or described in connection with FIGS. 2-6B. Such and other variations are contemplated to be within the scope of the present disclosure.

As mentioned in connection with FIG. 1, the third phase includes, after creating the PPI network, analysis using a Boolean Network Analysis (BNA) algorithm. Based on the expression levels of the proteins determined through this analysis, drug suggestions can be made. Proteins with altered expression levels can serve as input keywords in a drug database to identify available treatments.

Cellular molecules interact with one another in a structured manner, defining a regulatory network topology that describes cellular mechanisms. Genetic mutations alter these networks' pathways, generating complex disorders, such as autism spectrum disorder (ASD). Boolean models have assisted in understanding biological system dynamics, and various analytical tools for regulatory networks have been developed.

Boolean modeling is a graphical analytic approach used for analyzing qualitative models of biological systems and can be used to analyze protein-protein interaction networks. The analysis of the protein-protein interaction network can be used to identify the underlying etiology of the observed phenotype. The genetic mutations may be convergent with recognized signaling pathways, which have previously been implicated in the development of diseases. The disturbance in the activity of these genetic mutations may cause abnormal activation levels of critical proteins, such as β-catenin, MTORC1, RPS6, eIF4E, Cadherin, and SMAD, which regulate gene expression, translation, cell adhesion, shape, and migration. The varied functions of these proteins contribute to observed traits, and yet may reveal potential therapeutic options for them. Boolean network analysis may reveal abnormal activation levels of essential proteins such as β-catenin, MTORC1, RPS6, eIF4E, Cadherin, and SMAD. These proteins affect gene expression, translation, cell adhesion, shape, and migration.

After mapping the relations between the proteins, such as in the PPI network of FIG. 6, Boolean algebra, can be used to convert the relations and turn them to a Boolean model. The conjunction (AND), represented by (&); the disjunction (OR), represented by (|); and the negation (NOT), represented by (!), are the primary operators of Boolean algebra. Biological processes are non-linear, and certain genes may require extended activation periods for their regulator genes to become active. For example, it is typical to find statements in literature that say gene A increases or decreases the expression of gene B. This statement suggests that gene A can either enhance or suppress the activity of gene B, depending on whether gene B is affected by other signals that increase or decrease its activation. For this reason, the developers of SPIDDOR incorporated additional modulator operators to enhance the precision of defining various biological activities. The modulator operators define the activation and deactivation of Boolean functions in a network-based model. They are useful in simulating and analyzing complex systems. Simply put, these operators are used when there is no direct interaction between the genes, such as in the example of gene A increasing the expression of gene B.

The first operator is the threshold operator, represented as THR_(GENENAME) [n]. The threshold operator compares a vector of values to a certain set of values that partitions the multidimensional space with a hyper plane to classify the vector as false or true. The second operator is the modulator operator denoted by MOD_(GENENAME) [n]. This operator functions similarly to the THR operator but exclusively affects nodes that have modulation interactions within the Boolean functions of the network. The third operator is the ANY operator, denoted as ANY_GENENAME). The ANY operator determines whether a protein is activated or inactivated (Boolean functions true or false) in any of the last n iterations based on the conditions defined by the thresholds.

After discovering functional effect of proteins on the biological pathways, it can be found that the proteins activate, inhibit, and mediate molecule expression in signaling pathways. The protein-protein interaction (PPI) network (e.g., FIG. 6A and FIG. 6B) explains the protein relationships.

The use of dynamic evolution function in asynchronous mode can show the trajectory of molecules in the network according to the relations between them defined by Boolean equations. In an example, the output of one simulation for 100 time steps is a matrix encompassing (1 and 0). The dynamic evolution simulation of 2500 times for 100 steps yields 2500 1 and 0 matrices. In each of the 2500 matrices, rows represent network proteins, and columns indicate the 100 time steps. For example, when a mutation-like effect is introduced to the protein, its activity decreases by 50%. The total number of activations of the protein during the whole simulation is reduced to 30 out of 100, which is 50% of its original rate of activation in the normal state.

The Boolean analysis technique provides insight into the phenotype origin, pathophysiology, and therapy choices for diseased patients. The analysis technique can identify the genetic variants responsible for the disorder. This simplifies annotating these variants and incorporating them into biological pathways to reveal the cause. The Boolean network analysis method aids in identifying the most critical proteins that are influenced by genetic variations.

Where the protein-protein interaction network is a simple directed graph with no feedback loops included, the Boolean network analysis method may be the most appropriate graphical analytic approach, and it aids in revealing the hidden realities underneath the phenotype appearance. Other more advanced network analysis techniques may be employed in other situations, such as cyclic directed graphical models, dependency network models, or any type of graphical statistical probabilistic model that allows for cyclic direction in feedback loops in regulatory networks.

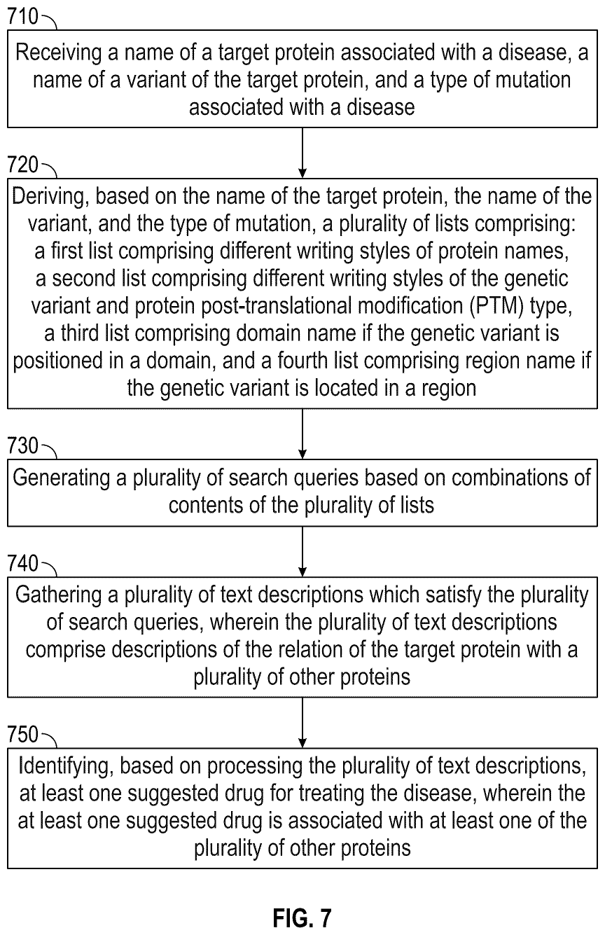

FIG. 7 is a flow diagram of an example operation, in accordance with aspects of the present disclosure.

At block 710, the operation involves receiving a name of a target protein associated with a disease, a name of a variant of the target protein, and a type of mutation associated with a disease.

At block 720, the operation involves deriving, based on the name of the target protein, the name of the variant, and the type of mutation, a plurality of lists that include: a first list containing different writing styles of protein names, a second list containing different writing styles of the genetic variant and protein post-translational modification (PTM) type, a third list comprising domain name if the genetic variant is positioned in a domain, and a fourth list comprising region name if the genetic variant is located in a region.

At block 730, the operation involves generating a plurality of search queries based on combinations of contents of the plurality of lists.

At block 740, the operation involves gathering a plurality of text descriptions which satisfy the plurality of search queries, wherein the plurality of text descriptions include descriptions of the relation of the target protein with a plurality of other proteins.

At block 750, the operation involves identifying, based on processing the plurality of text descriptions, at least one suggested drug for treating the disease, where the at least one suggested drug is associated with at least one of the plurality of other proteins.

FIG. 7 and the description above are merely an example and variations are contemplated to be within the scope of the present disclosure. In various embodiments, the operation may include other blocks not shown or described in connection with FIG. 7. In various embodiments, the operation may not include every block shown or described in connection with FIG. 7. In various, the blocks may be performed in a different order than as shown or described in connection with FIG. 7. Such and other variations are contemplated to be within the scope of the present disclosure.

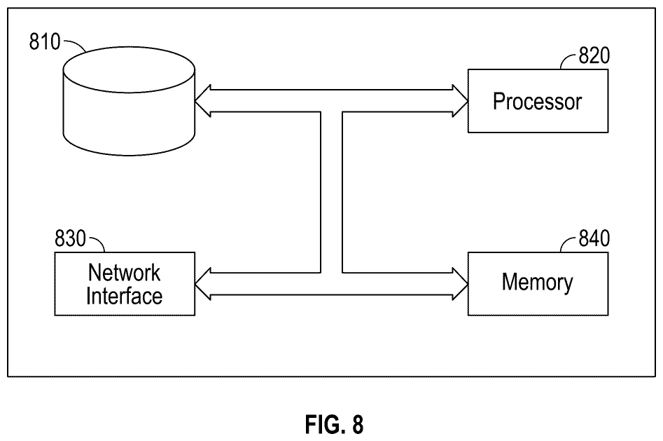

FIG. 8 is a block diagram of an example of computing components that may be used to perform any of the operations or any aspects of the operations described herein, including the aspects and operations described in connection with any of FIGS. 1-7.

The computing components include an electronic storage 810, a processor 820, a memory 840, and a network interface 830. The various components may be communicatively coupled with each other. The processor 820 may be and may include any type of processor, such as a single-core central processing unit (CPU), a multi-core CPU, a microprocessor, a digital signal processor (DSP), a System-on-Chip (SoC), or any other type of processor. The memory 840 may be a volatile type of memory, e.g., RAM, or a non-volatile type of memory, e.g., NAND flash memory. The memory 840 includes processor-readable instructions that are executable by the processor 820 to cause the system to perform various operations, including those mentioned herein, such as the operations described in connection with of FIGS. 1-7.

The electronic storage 810 may be and include any type of electronic storage used for storing data, such as hard disk drive, solid state drive, and/or optical disc, among other types of electronic storage. The electronic storage 810 stores processor-readable instructions for causing the system to perform its operations and stores data associated with such operations, such as storing data relating to any of the sequences, clusters, or confidence scores, among other data. The network interface 830 may implement networking technologies, such as Ethernet, Wi-Fi, and/or other wireless networking technologies.

The components shown in FIG. 8 are merely examples, and persons skilled in the art will understand that a system includes other components not illustrated and may include multiples of any of the illustrated components. Such and other embodiments are contemplated to be within the scope of the present disclosure.

The following are hereby incorporated by reference herein in their entirety:
Nezamuldeen, L. and M. S. Jafri. 2023. Protein-Protein Interaction Network Extraction Using Text Mining Methods Adds Insight into Autism Spectrum Disorder. Biology. 12 (10): 1344.
Nezamuldeen, L. and M. S. Jafri. 2024. Boolean Modeling of Biological Network Applied to Protein-Protein Interaction Network of Autism Patients. Biology. 13 (8): 606.
Nezamuldeen, L. and M. S. Jafri. 2024. Text Mining to Understand Disease-Causing Gene Variants. Knowledge. 4 (3), 422-443.

The embodiments disclosed herein are examples of the disclosure and may be embodied in various forms. For instance, although certain embodiments herein are described as separate embodiments, each of the embodiments herein may be combined with one or more of the other embodiments herein. Specific structural and functional details disclosed herein are not to be interpreted as limiting, but as a basis for the claims and as a representative basis for teaching one skilled in the art to variously employ the present disclosure in virtually any appropriately detailed structure. Like reference numerals may refer to similar or identical elements throughout the description of the figures.

The phrases “in an embodiment,” “in embodiments,” “in various embodiments,” “in some embodiments,” or “in other embodiments” may each refer to one or more of the same or different embodiments in accordance with the present disclosure. A phrase in the form “A or B” means “(A), (B), or (A and B).” A phrase in the form “at least one of A, B, or C” means “(A); (B); (C); (A and B); (A and C); (B and C); or (A, B, and C).”

The systems, devices, and/or servers described herein may utilize one or more processors to receive various information and transform the received information to generate an output. The processors may include any type of computing device, computational circuit, or any type of controller or processing circuit capable of executing a series of instructions that are stored in a memory. The processor may include multiple processors and/or multicore central processing units (CPUs) and may include any type of device, such as a microprocessor, graphics processing unit (GPU), digital signal processor, microcontroller, programmable logic device (PLD), field programmable gate array (FPGA), or the like. The processor may also include a memory to store data and/or instructions that, when executed by the one or more processors, causes the one or more processors to perform one or more methods and/or algorithms.

Any of the herein described methods, programs, algorithms or codes may be converted to, or expressed in, a programming language or computer program. The terms “programming language” and “computer program,” as used herein, each include any language used to specify instructions to a computer, and include (but is not limited to) the following languages and their derivatives: Assembler, Basic, Batch files, BCPL, C, C+, C++, Delphi, Fortran, Java, JavaScript, machine code, operating system command languages, Pascal, Perl, PL1, Python, scripting languages, Visual Basic, metalanguages which themselves specify programs, and all first, second, third, fourth, fifth, or further generation computer languages. Also included are database and other data schemas, and any other meta-languages. No distinction is made between languages which are interpreted, compiled, or use both compiled and interpreted approaches. No distinction is made between compiled and source versions of a program. Thus, reference to a program, where the programming language could exist in more than one state (such as source, compiled, object, or linked) is a reference to any and all such states. Reference to a program may encompass the actual instructions and/or the intent of those instructions.

It should be understood that the foregoing description is only illustrative of the present disclosure. Various alternatives and modifications can be devised by those skilled in the art without departing from the disclosure. Accordingly, the present disclosure is intended to embrace all such alternatives, modifications and variances. The embodiments described with reference to the attached drawing figures are presented only to demonstrate certain examples of the disclosure. Other elements, steps, methods, and techniques that are insubstantially different from those described above and/or in the appended claims are also intended to be within the scope of the disclosure.

What is claimed:

1. A processor-implemented method comprising:
receiving a name of a target protein associated with a disease, a name of a variant of the target protein, and a type of mutation associated with a disease;
deriving, based on the name of the target protein, the name of the variant, and the type of mutation, a plurality of lists comprising:
a first list comprising different writing styles of protein names,
a second list comprising different writing styles of the genetic variant and protein post-translational modification (PTM) type,
a third list comprising domain name if the genetic variant is positioned in a domain, and
a fourth list comprising region name if the genetic variant is located in a region;
generating a plurality of search queries based on combinations of contents of the plurality of lists;
gathering a plurality of text descriptions which satisfy the plurality of search queries, wherein the plurality of text descriptions comprises descriptions of the relation of the target protein with a plurality of other proteins; and
identifying, based on processing the plurality of text descriptions, at least one suggested drug for treating the disease, wherein the at least one suggested drug is associated with at least one of the plurality of other proteins.

2. The processor-implemented method of claim 1, further comprising processing the plurality of text descriptions by applying a neural network to extract sentences containing protein-protein interactions (PPI).

3. The processor-implemented method of claim 2, wherein the neural network comprises three layers of Bidirectional Long Short-Term Memory (BILSTM), recurrent neural network (RNN) cells, and a BioWordVec pretrained word embedding layer to extract positive sentences that comprises protein-protein interaction.

4. The processor-implemented method of claim 2, further comprising, in the extracted positive sentences, labeling the name of the target protein name with a first indicator and labeling the name of the other proteins that interact with the target protein with a second indicator.

5. The processor-implemented method of claim 4, wherein the labeling is performed by applying a named entity recognition (NER) model and using a conditional random fields (CRF) algorithm.

6. The processor-implemented method of claim 4, wherein the first indicator is a letter “P” and the second indicator is a letter “O”.

7. The processor-implemented method of claim 4, further comprising identifying, based on the first indicators and the second indicators in the labeled sentences, shortest paths between separate proteins described in the extracted sentences.

8. The processor-implemented method of claim 7, further comprising extracting relationship words in the extracted sentences relating to relationships of the separate proteins, wherein the extracting uses predetermined patterns.

9. The processor-implemented method of claim 8, further comprising creating a PPI network based on the separate proteins described in the extracted sentences and based on the relationship words in the extracted sentences relating to relationships of the separate proteins.

10. The processor-implemented method of claim 1, wherein the identifying the at least one suggested drug for treating the disease comprises:
analyzing expression levels of the plurality of other proteins;
identifying at least one other protein of the plurality of other proteins having altered expression levels; and
identifying the at least one suggested drug for treating the disease based on the at least one other protein having altered expression levels.

11. The processor-implemented method of claim 1, wherein the at least one suggested drug is not associated with the protein associated with the disease.

12. The processor-implemented method of claim 1, wherein the variant of the protein comprises an amino acid substitution.

13. The processor-implemented method of claim 12, wherein the amino acid substitution is located at a phosphorylation, acetylation, methylation, sumoylation, or ubiquitination site.

14. The processor-implemented method of claim 1, wherein the variant of the protein comprises a truncated protein.

15. The processor-implemented method of claim 1, wherein the protein-protein interaction (PPI) network identifies an abnormal protein-protein interaction.

16. A system comprising:
one or more processors; and
one or more processor-readable medium having stored thereon instructions which, when executed by the one or more processors, cause the system at least to perform:
receiving a name of a target protein associated with a disease, a name of a variant of the target protein, and a type of mutation associated with a disease;
deriving, based on the name of the target protein, the name of the variant, and the type of mutation, a plurality of lists comprising:
a first list comprising different writing styles of protein names,
a second list comprising different writing styles of the genetic variant and protein post-translational modification (PTM) type,
a third list comprising domain name if the genetic variant is positioned in a domain, and
a fourth list comprising region name if the genetic variant is located in a region;
generating a plurality of search queries based on combinations of contents of the plurality of lists;
gathering a plurality of text descriptions which satisfy the plurality of search queries, wherein the plurality of text descriptions comprises descriptions of the relation of the target protein with a plurality of other proteins; and
identifying, based on processing the plurality of text descriptions, at least one suggested drug for treating the disease, wherein the at least one suggested drug is associated with at least one of the plurality of other proteins.

17. A processor-readable medium having stored thereon instructions which, when executed by one or more processors of a system, cause the system at least to perform:
receiving a name of a target protein associated with a disease, a name of a variant of the target protein, and a type of mutation associated with a disease;
deriving, based on the name of the target protein, the name of the variant, and the type of mutation, a plurality of lists comprising:
a first list comprising different writing styles of protein names,
a second list comprising different writing styles of the genetic variant and protein post-translational modification (PTM) type,
a third list comprising domain name if the genetic variant is positioned in a domain, and
a fourth list comprising region name if the genetic variant is located in a region;
generating a plurality of search queries based on combinations of contents of the plurality of lists;
gathering a plurality of text descriptions which satisfy the plurality of search queries, wherein the plurality of text descriptions comprises descriptions of the relation of the target protein with a plurality of other proteins; and
identifying, based on processing the plurality of text descriptions, at least one suggested drug for treating the disease, wherein the at least one suggested drug is associated with at least one of the plurality of other proteins.
